(19) United States
(12) Patent Application Publication
(10) Pub. No.: US 2023/0360216 A1
(43) Pub. Date: Nov. 9, 2023

(54) SYSTEMS AND METHODS FOR DETECTING PERFUSION IN SURGERY

(71) Applicant: Covidien LP, Mansfield, MA (US)

(72) Inventors: James D. Allen, IV, Broomfield, CO (US); Dori Peleg, Kiryat Bialik (IL); Teresa A. Whitman, Dayton, MN (US); Nicole Kirchhof, Minnetonka, MN (US); William J. Peine, Ashland, MA (US); Eugene A. Stellon, JR., Burlington, CT (US)

(21) Appl. No.: 17/735,430

(22) Filed: May 3, 2022

(51) Int. Cl. G06T 7/00; A61B 5/026; A61B 5/00

(52) U.S. Cl. CPC G06T 7/0016; A61B 5/026; A61B 5/7264; A61B 5/7425; G06T 2207/10012; G06T 2207/20081; G06T 2207/30028; A61B 2090/371

(57) ABSTRACT

A surgical system for detecting perfusion includes at least one surgical camera and a computing device. The at least one surgical camera is configured to obtain image data of tissue at a surgical site including first image data and second image data that is temporally-spaced relative to the first image data. The computing device is configured to receive the image data from the at least one surgical camera and includes a non-transitory computer-readable storage medium storing instructions configured to cause the computing device to detect differences between the first and second image data, determine a level of perfusion in the tissue based on the detected differences between the first and second image data, and provide an output indicative of the determined level of perfusion in the tissue.

### FIELD

This disclosure relates to surgery and, more particularly, to systems and methods for detecting perfusion in surgery.

### BACKGROUND

Adequate perfusion, or blood supply, at a surgical site is important in order to increase the likelihood of faster and favorable post-surgery healing. For example, one of the main prerequisites for favorable anastomotic healing in low anterior resection (LAR) surgery is to ensure that adequate perfusion is present. Poor perfusion can lead to a symptomatic anastomotic leak (AL) after LAR surgery. AL's after LAR surgery are associated with a high level of morbidity and a leak-related mortality rate of as high as 39%.

### SUMMARY

Any or all of the aspects and features detailed herein, to the extent consistent, may be used in conjunction with any or all of the other aspects and features detailed herein.

Provided in accordance with aspects of this disclosure is a surgical system for detecting perfusion. The surgical system includes at least one surgical camera and a computing device. The at least one surgical camera is configured to obtain image data of tissue at a surgical site including first image data and second image data that is temporally-spaced relative to the first image data. The computing device is configured to receive the image data from the at least one surgical camera and includes a non-transitory computer-readable storage medium storing instructions configured to cause the computing device to detect differences between the first and second image data, determine a level of perfusion in the tissue based on the detected differences between the first and second image data, and provide an output indicative of the determined level of perfusion in the tissue.

In an aspect of this disclosure, computing device is further caused to amplify the detected differences between the first and second image data. In such aspects, the level of perfusion in the tissue may be determined based on the amplified detected differences between the first and second image data.

In another aspect of this disclosure, the at least one surgical camera includes first and second surgical cameras such that the image data is stereographic image data from the first and second surgical cameras.

In still another aspect of this disclosure, the surgical system further includes an ultraviolet light source configured to illuminate the tissue at the surgical site such that the image data includes ultraviolet-enhanced image data.

In yet another aspect of this disclosure, the image data is video image data, infrared image data, thermal image data, or ultrasound image data.

In still yet another aspect of this disclosure, the level of perfusion is determined by a machine learning algorithm of the computing device. The machine learning algorithm, in such aspects, may be configured to receive the detected differences between the first and second image data and determine the level of perfusion based on the detected differences between the first and second image data. Alternatively, the machine learning algorithm, in such aspects, may be configured to receive the first and second image data, detect the differences between the first and second image data, and determine the level of perfusion based on the detected differences between the first and second image data.

In another aspect of this disclosure, the output indicative of the determined level of perfusion in the tissue includes a visual indicator on a display configured to display a video feed of the surgical site.

In another aspect of this disclosure, the output indicative of the determined level of perfusion in the tissue includes a visual overlay, on a display, over a video feed of the surgical site.

A method for detecting perfusion in surgery in accordance with aspects of this disclosure includes obtaining, from at least one surgical camera, first image data of tissue at a surgical site; obtaining, from the at least one surgical camera, second image data of the tissue at the surgical site that is temporally-spaced relative to the first image data; detecting differences between the first and second image data; determining a level of perfusion based on the detected differences between the first and second image data; and providing an output indicative of the determined level of perfusion.

In an aspect of this disclosure, the method further includes amplifying the detected differences between the first and second image data before determining the level of perfusion in the tissue such that the level of perfusion in the tissue is determined based on the amplified detected differences between the first and second image data.

In another aspect of this disclosure, the at least one surgical camera includes first and second surgical cameras such that obtaining the first and second image data includes obtaining first and second stereographic image data, respectively.

In still another aspect of this disclosure, the method further includes illuminating the tissue at the surgical site with ultraviolet light such that the first image data is ultraviolet-enhanced image data and the second image data is ultraviolet-enhanced image data.

In yet another aspect of this disclosure, obtaining each of the first and second image data includes obtaining video image data, infrared image data, thermal image data, or ultrasound image data.

In still yet another aspect of this disclosure, determining the level of perfusion based on the detected differences between the first and second image data includes implementing a machine learning algorithm. In such aspects, the machine learning algorithm may be configured to receive the detected differences between the first and second image data and determine the level of perfusion based on the detected differences between the first and second image data. Alternatively, in such aspects, the machine learning algorithm may be configured to receive the first and second image data, to detect the differences between the first and second image data, and to determine the level of perfusion based on the detected differences between the first and second image data.

In another aspect of this disclosure, providing the output indicative of the determined level of perfusion in the tissue includes providing a visual indicator on a display configured to display a video feed of the surgical site.

In another aspect of this disclosure, providing the output indicative of the determined level of perfusion in the tissue includes providing a visual overlay, on a display, over a video feed of the surgical site.

### BRIEF DESCRIPTION OF DRAWINGS

The above and other aspects and features of this disclosure will become more apparent in view of the following detailed description when taken in conjunction with the accompanying drawings wherein like reference numerals identify similar or identical elements.

FIG. 1 is a perspective view of a surgical system provided in accordance with aspects of this disclosure;

FIGS. 2A and 2B are anatomical views illustrating a low anterior resection (LAR) surgical procedure;

FIG. 3 is a schematic illustration of the surgical system of FIG. 1 in use during a surgical procedure, e.g., a LAR, in accordance with aspects of this disclosure;

FIG. 4 is a schematic illustration of the surgical system of FIG. 1 in use during a surgical procedure, e.g., a LAR, in accordance with other aspects this disclosure;

FIG. 5 is a schematic illustration of the surgical system of FIG. 1 in use during a surgical procedure, e.g., a LAR, in accordance with still other aspects of this disclosure;

FIG. 6 is a flow diagram of a method in accordance with aspects of this disclosure;

FIG. 7 is a logic diagram of a machine learning algorithm in accordance with the present disclosure;

FIG. 8 is a graphical representation of a display provided in accordance with this disclosure shown displaying a perfusion indicator and video image data; and

FIG. 9 is a graphical representation of a display provided in accordance with this disclosure shown displaying perfusion data overlaid over video image data.

### DETAILED DESCRIPTION

This disclosure provides systems and methods for detecting perfusion during surgery. Although detailed herein with respect to a low anterior resection (LAR) surgical procedure, it is understood that the present disclosure is equally applicable for use in any other suitable surgical procedure.

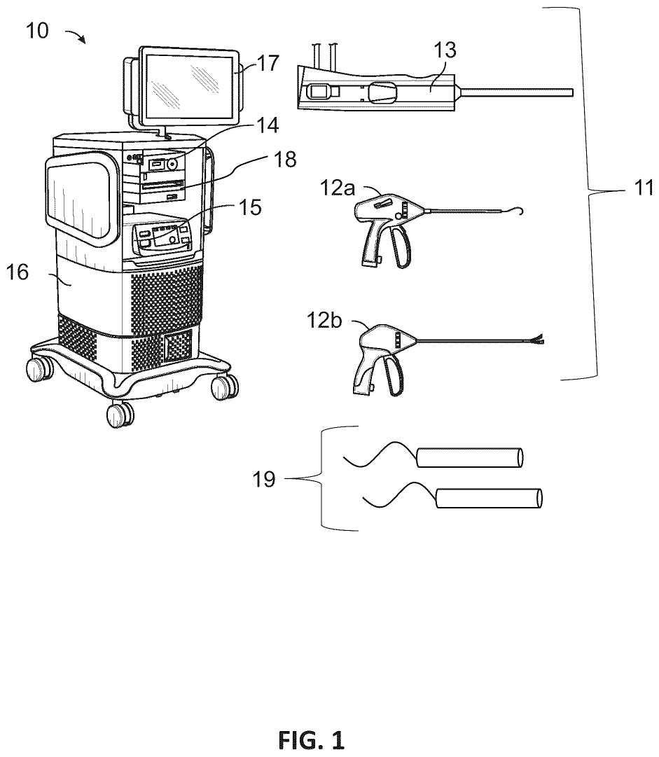

Referring to FIG. 1, a surgical system 10 provided in accordance with this disclosure is shown including at least one surgical instrument 11, a surgical controller 14 configured to connect to one or more of the at least one surgical instrument 11, a surgical generator 15 configured to connect to one or more of the at least one surgical instrument 11, a control tower 16 housing the surgical controller 14 and the surgical generator 15, and a display 17 disposed on control tower 16 and configured to output, for example, video and/or other imaging data from one or more of the at least one surgical instrument 11 and to display operating parameter data, feedback data, etc. from one or more of the at least one surgical instrument 11 and/or generator 15. Display 17 and/or a separate user interface (not shown) may be provided to enable user input, e.g., via a keyboard, mouse, touch-screen GUI, etc.

The at least one surgical instrument 11 may include, for example, a first surgical instrument 12a for manipulating and/or treating tissue, a second surgical instrument 12b for manipulating and/or treating tissue, and/or a third surgical instrument 13 for visualizing and/or providing access to a surgical site. The first and/or second surgical instruments 12a, 12b may include: energy-based surgical instruments for grasping, sealing, and dividing tissue such as, for example, an electrosurgical forceps, an ultrasonic dissector, etc.; energy-based surgical instruments for tissue dissection, resection, ablation and/or coagulation such as, for example, an electrosurgical pencil, a resection wire, an ablation (microwave, radiofrequency, cryogenic, etc.) device, etc.; mechanical surgical instruments configured to clamp and close tissue such as, for example, a surgical stapler, a surgical clip applier, etc.; mechanical surgical instruments configured to facilitate manipulation and/or cutting of tissue such as, for example, a surgical grasper, surgical scissors, a surgical retractor, etc.; and/or any other suitable surgical instruments. Although first and second surgical instruments 12a, 12b are shown in FIG. 1, greater or fewer of such instruments 12a, 12b are also contemplated.

The third surgical instrument 13 may include, for example, an endoscope or other suitable surgical camera to enable visualizing into a surgical site. The third surgical instrument 13 may additionally or alternatively include one or more access channels to enable insertion of first and second surgical instruments 12a, 12b, aspiration/irrigation, insertion of any other suitable surgical tools, etc. The third surgical instrument 13 may be coupled, via wired or wireless connection, to controller 14 (and/or computing device 18) for processing the video data for displaying the same on display 17. Although one third surgical instrument 13 is shown in FIG. 1, more of such instruments 13 are also contemplated.

Surgical system 10, in aspects, also includes at least one surgical camera 19 such as, for example, one or more surgical cameras 19 configured to collect imaging data from a surgical site, e.g., using still picture imaging, video imaging, thermal imaging, infrared imaging, ultrasound imaging, etc. In aspects, the at least one surgical camera 19 is provided in addition to or as an alternative to the one or more third surgical instruments 13. In other aspects, third surgical instrument(s) 13 provide the functionality of surgical camera(s) 19. Surgical camera(s) 19 is coupled, via wired or wireless connection, to computing device 18 for providing the image data thereto, e.g., in real time, to enable the computing device 18 to process the received image data, e.g., in real time, and provide a suitable output based thereon, as detailed below.

Continuing with reference to FIG. 1, surgical system 10 further includes a computing device 18, which is in wired or wireless communication with one or more of the at least one surgical instrument 11, surgical controller 14, generator 15, display 17, and/or surgical camera 19. Computing device 18 is capable of receiving data, e.g., activation data, actuation data, feedback data, etc., from first and/or second instruments 12a, 12b, video data from the one or more third instrument 13, and/or the image data from the one or more surgical cameras 19. Computing device 18 may process some or all of the received data substantially at the same time upon reception of the data, e.g., in real time. Further, computing device 18 may be capable of providing desired parameters to and/or receiving feedback data from first and/or second instruments 12a, 12b, surgical controller 14, surgical generator 15 (for implementation in the control of surgical instruments 12a, 12b, for example), and/or other suitable devices in real time to facilitate feedback-based control of a surgical operation and/or output of suitable display information for display on display 17, e.g., beside, together with, as an overlay on, etc., the video feed from third instrument 13. Computing device 18 is described in greater detail below.

Although computing device 18 is shown as a single unit disposed on control tower 16, computing device 18 may include one or more local, remote, and/or virtual computers that communicate with one another and/or the other devices of surgical system 10 using any suitable communication network based on wired or wireless communication protocols. Computing device 18, more specifically, may include, by way of non-limiting examples, one or more: server computers, desktop computers, laptop computers, notebook computers, Internet appliances, mobile smartphones, tablet computers, personal digital assistants, embedded computers, and the like. Computing device 18 further includes an operating system configured to perform executable instructions. The operating system is, for example, software, including programs and data, which manages the device's hardware and provides services for execution of applications. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, Novell® NetWare®, and the likes. In aspects, the operating system may be provided by cloud computing.

Computing device 18 includes a storage implemented as one or more physical apparatus used to store data or programs on a temporary or permanent basis. The storage may be volatile memory, which requires power to maintain stored information, or non-volatile memory, which retains stored information even when the computing device 18 is not powered on. In aspects, the non-volatile memory includes flash memory, dynamic random-access memory (DRAM), ferroelectric random-access memory (FRAM), and phase-change random access memory (PRAM). In aspects, the storage may include, by way of non-limiting examples, CD-ROMs, DVDs, flash memory devices, magnetic disk drives, magnetic tapes drives, optical disk drives, solid-state drive, universal serial bus (USB) drive, and cloud computing-based storage. In aspects, the storage may be any combination of storage media such as those disclosed herein.

Computing device 18 further includes a processor, an extension, an input/output device, and a network interface, although additional or alternative components are also contemplated. The processor executes instructions which implement tasks or functions of programs. When a user executes a program, the processor reads the program stored in the storage, loads the program on the RAM, and executes instructions prescribed by the program. Although referred to herein in the singular, it is understood that the term processor includes multiple similar or different processes locally, remotely, or both locally and remotely distributed.

The processor of computing device 18 may include a field programmable gate array (FPGA), a digital signal processor (DSP), a central processing unit (CPU), a graphical processing unit (GPU), a microprocessor, application specific integrated circuit (ASIC), and combinations thereof, each of which includes electronic circuitry within a computer that carries out instructions of a computer program by performing the basic arithmetic, logical, control and input/output (I/O) operations specified by the instructions. Those skilled in the art will appreciate that the processor may be substituted for by using any logic processor (e.g., control circuit) adapted to execute algorithms, calculations, and/or set of instructions described herein.

In aspects, the extension may include several ports, such as one or more USBs, IEEE 1394 ports, parallel ports, and/or expansion slots such as peripheral component interconnect (PCI) and PCI express (PCIe). The extension is not limited to the list but may include other slots or ports that can be used for appropriate purposes. The extension may be used to install hardware or add additional functionalities to a computer that may facilitate the purposes of the computer. For example, a USB port can be used for adding additional storage to the computer and/or an IEEE 1394 may be used for receiving moving/still image data.

The network interface is used to communicate with other computing devices, wirelessly or via a wired connection following suitable communication protocols. Through the network interface, computing device 18 may transmit, receive, modify, and/or update data from and to an outside computing device, server, or clouding space. Suitable communication protocols may include, but are not limited to, transmission control protocol/internet protocol (TCP/IP), datagram protocol/internet protocol (UDP/IP), and/or datagram congestion control protocol (DCCP). Wireless communication may be achieved via one or more wireless configurations, e.g., radio frequency-embedded millimeter wave transvers optical, Wi-Fi, Bluetooth (an open wireless protocol for exchanging data over short distances, using short length radio waves, from fixed and mobile devices, creating personal area networks (PANs), ZigBee® (a specification for a suite of high level communication protocols using small, low-power digital radios based on the IEEE 122.15.4-2003 standard for wireless personal area networks (WPANs)).

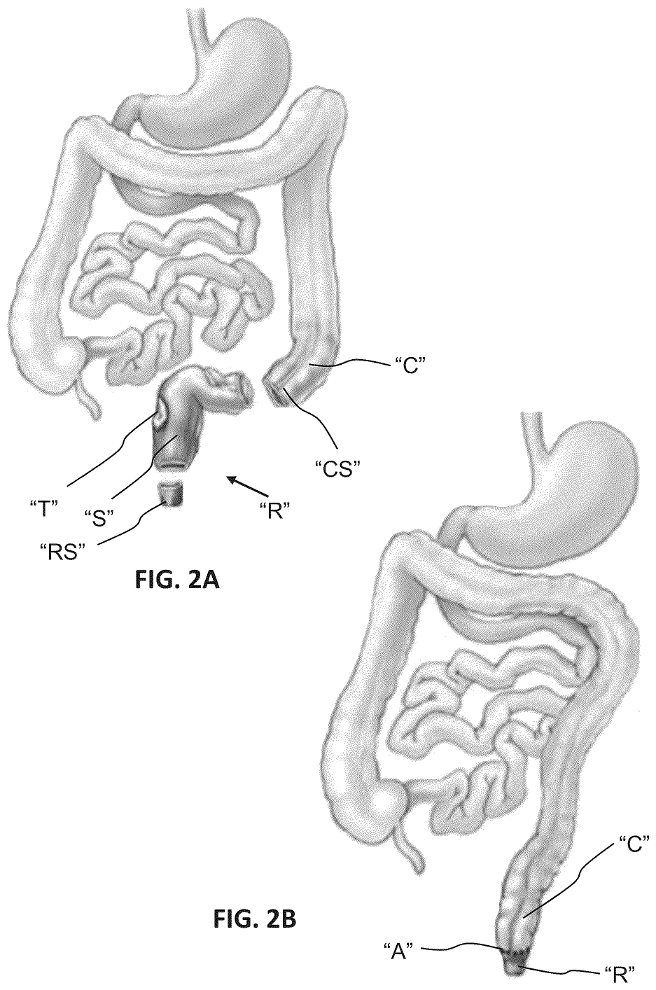

Turning to FIGS. 2A and 2B, low anterior resection (LAR) surgical procedures are typically performed to treat diseases of the rectum “R” such as a cancerous rectal tumor “T.” LAR surgical procedures can be performed laparoscopically or in any other suitable manner. During an LAR surgical procedure, a section “S” of the rectum “R” including the diseased portion or, in certain instances, the entirety of the rectum “R,” is removed (with sufficient margins on either side). Once the section “S” is removed, the rectal and colonic stumps “RS” and “CS,” respectively, are joined via an anastomosis “A” to reconnect the remaining portion of the rectum “R” to the colon “C.” During such an LAR surgical procedure, it is important to assess the level of perfusion to ensure adequate blood supply to the rectal and colonic stumps “RS” and “CS,” respectively, prior to the anastomosis “A,” as well as to ensure adequate blood supply to the rectum “R” and colon “C” after the anastomosis “A.” Adequate blood supply is an important factor in promoting faster and favorable post-surgery healing as well as to reduce the likelihood of an anastomotic leak (AL).

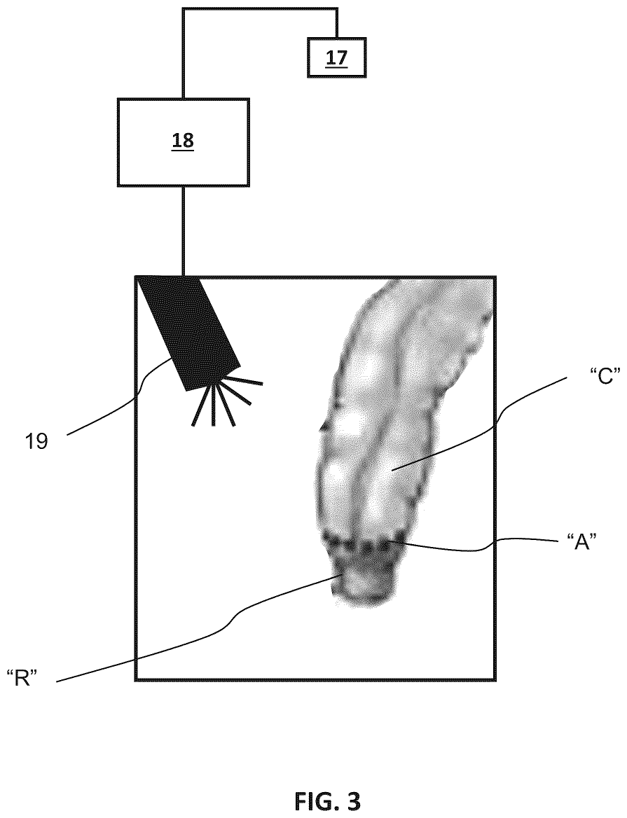

Referring to FIG. 3, in aspects, surgical camera 19 may be utilized to collect image data from the surgical site during an LAR surgical procedure (or other surgical procedure) such as, for example, video image data, thermal image data, infrared image data, ultrasound image data, etc. The image data collected by surgical camera 19 is transmitted to computing device 18 to enable processing of the image data as a function of time to determine a level of perfusion at the surgical site, e.g., within the field of view of surgical camera such as, for example of the rectum “R” and colon “C.” An output indicating the level of perfusion at the surgical site may be displayed on display 17 or otherwise provided in real time to facilitate performing the LAR surgical procedure (or other surgical procedure). The image data collected by surgical camera 19 may additionally or alternatively be processed and output as a video feed on display 17, although a separate camera for providing the video feed on display may also be utilized, e.g., third surgical instrument 13 (FIG. 1) or another surgical camera 19.

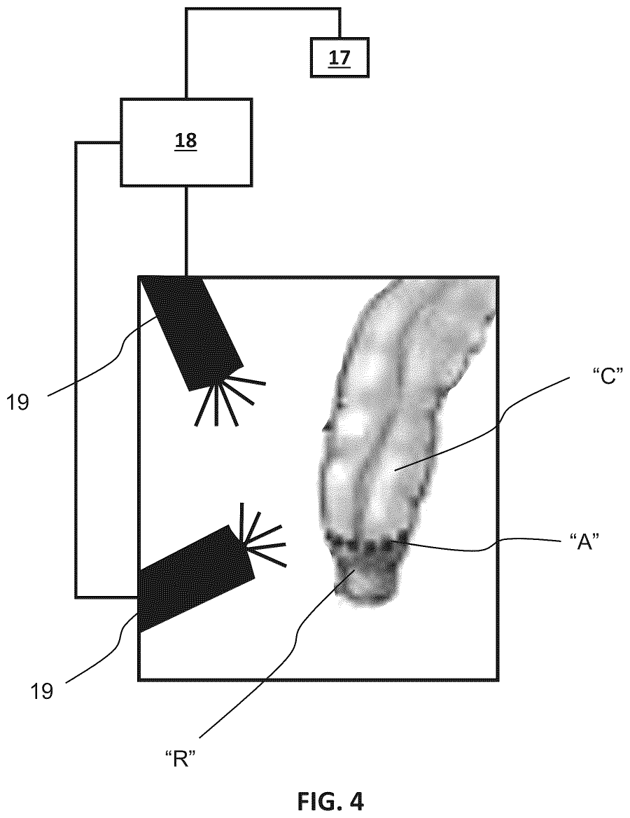

With reference to FIG. 4, in aspects, at least two surgical cameras 19 may be utilized to collect stereographic image data from the surgical site during an LAR surgical procedure (or other surgical procedure) such as, for example, stereographic video image data, stereographic thermal image data, stereographic infrared image data, stereographic ultrasound image data, etc. The stereographic image data collected by surgical camera 19 is transmitted to computing device 18 to enable processing of the stereographic image data as a function of time to determine a level of perfusion at the surgical site, e.g., within the field of view of surgical camera such as, for example of the rectum “R” and colon “C.” An output indicating the level of perfusion at the surgical site may be displayed on display 17 or otherwise provided in real time to facilitate performing the LAR surgical procedure (or other surgical procedure). The image data collected by either or both surgical cameras 19 may additionally or alternatively be processed and output as a video feed on display 17, although a separate camera for providing the video feed on display may also be utilized, e.g., third surgical instrument 13 (FIG. 1) or another surgical camera 19. In aspects where the image data from both surgical cameras 19 is utilized, the video feed provided on display 17 may be a three-dimensional (3D) video feed or a video feed including a 3D overlay to highlight perfusion within the field of view.

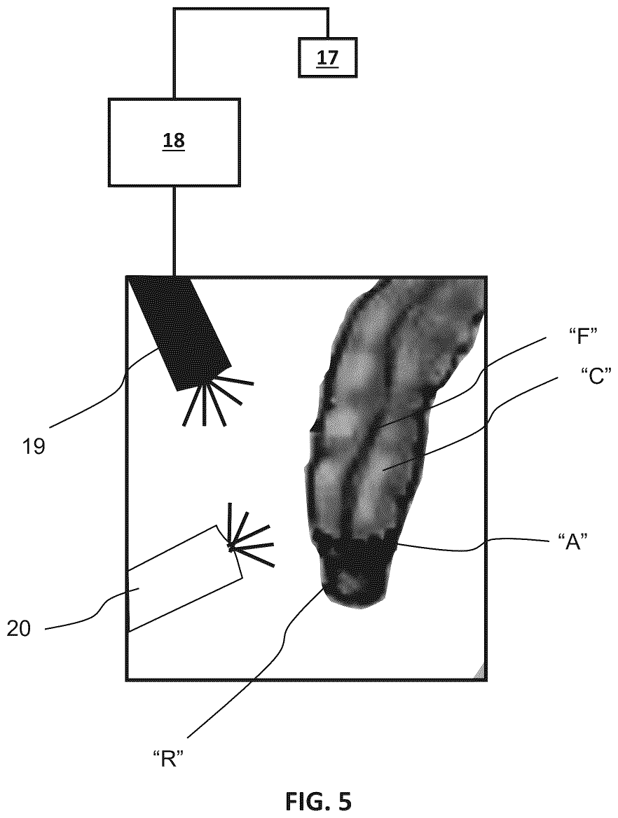

As shown in FIG. 5, in aspects, fluorescent markers or dye “F” can be injected in the patient's blood stream to facilitate the collection of ultraviolet-enhanced image data from the surgical site during an LAR surgical procedure (or other surgical procedure). More specifically, an ultraviolet light source 20 may utilized to illuminate at least a portion of the field of view of one or more surgical cameras 19 such as, for example, the rectum “R” and colon “C.” As a result, the one or more surgical cameras 19 are able to collect ultraviolet-enhanced image data resulting from the activation of the fluorescent markers or dye “F” within the blood stream via the ultraviolet light from ultraviolet light source 20. In aspects, the ultraviolet-enhanced imaging data may be obtained using a single surgical camera 19, similarly as detailed above with respect to FIG. 3, or stereographically using multiple surgical cameras 19, similarly as detailed above with respect to FIG. 4. The ultraviolet-enhanced image data collected by surgical camera 19 is transmitted to computing device 18 to enable processing of the image data as a function of time to determine a level of perfusion at the surgical site, e.g., within the field of view of surgical camera such as, for example of the rectum “R” and colon “C.” An output indicating the level of perfusion at the surgical site may be displayed on display 17 or otherwise provided in real time to facilitate performing the LAR surgical procedure (or other surgical procedure). The ultraviolet-enhanced image data collected by surgical camera(s) 19 may additionally or alternatively be processed and output as a video feed on display 17, although a separate camera for providing the video feed on display 17 may also be utilized, e.g., third surgical instrument 13 (FIG. 1) or another surgical camera 19. Additionally or alternatively, the ultraviolet-enhanced image data may be output for display as an overlay on the video feed to highlight perfusion within the field of view.

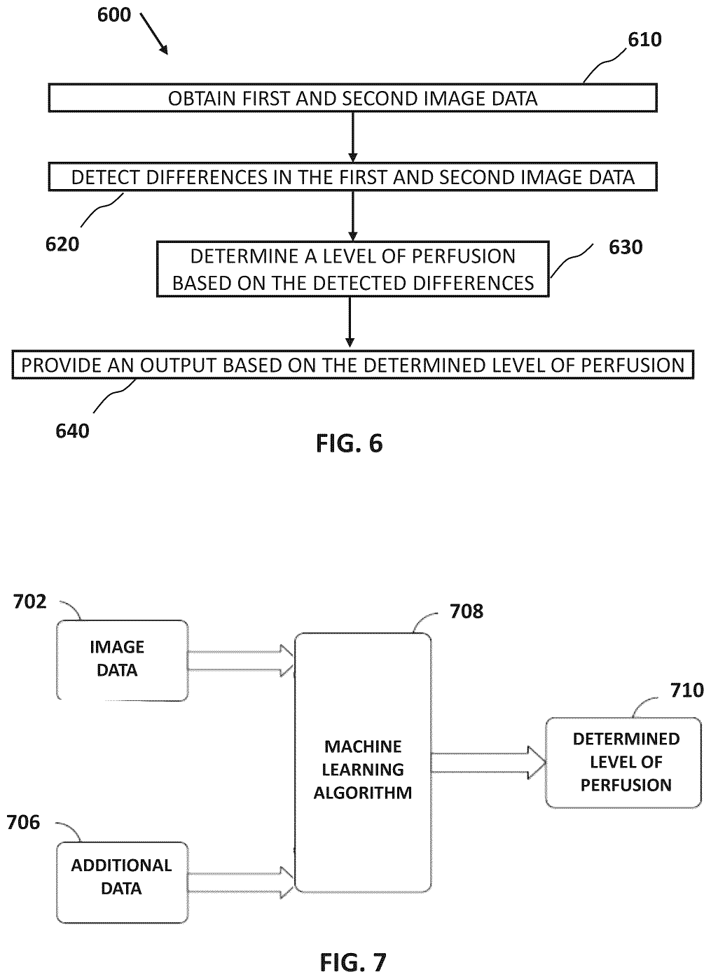

Turning to FIG. 6, in conjunction with FIGS. 3-5, as noted above, image data may be processed as a function of time to determine a level of perfusion at the surgical site. A method for processing the image data as a function of time to determine a level of perfusion at the surgical site in accordance with this disclosure is shown as method 600. Method 600 may be implemented by computing device 18 (FIG. 1) and/or any other suitable computing device. Initially, at 610, at least first and second image data is obtained. The first and second image data may be, for example and as detailed above, video image data, thermal image data, infrared image data, ultrasound image data, etc. and may be monographic image data, stereographic image data, and/or ultraviolet-enhanced image data. The first and second image data are temporally spaced such that for example the first image data corresponds to a first time and the second image data corresponds to a second, different time. Although detailed herein with respect to first and second image data, it is understood that additional temporally-spaced image data may also be utilized and/or that method 600 may be performed repeatedly on additional image data to provide a real-time output, wherein each iteration of method 600 includes at least first and second image data.

As indicated at 620, differences between the temporally spaced first and second image data are detected. For example, differences in pixel color and/or intensity between the first image data and the second image data may be detected. As another example, movement and/or change in the size (expansion, contraction, etc.) of identified structures between the first image data and the second image data may be detected. In aspects, these differences are amplified so as to exaggerate, for example, the differences in pixel colors and/or intensities between the first image data and the second image data, and/or movements and/or size changes of identified structures between the first image data and the second image data. This amplification may be performed such as detailed in U.S. Pat. Nos. 9,805,475 and/or 9,811,901, each of which is hereby incorporated herein by reference. In other aspects, the differences are not amplified. In either configuration, the differences may be further processed to facilitate analysis.

At 630, a level of perfusion is determined based on the detected differences between the temporally spaced first and second image data (whether or not amplified or processed in any other suitable manner). More specifically, the detected differences between the temporally spaced first and second image data enables the detection of pulsations (expansions and contractions) of tissue such as blood vessels within or on the surface of tissue, e.g., the rectum “R” and colon “C” (see FIG. 3-5). These pulsations indicate blood flow through the blood vessels as the heart beats and, thus, can be evaluated in density and/or magnitude to determine a level of perfusion. Additionally or alternatively, the detected differences between the temporally spaced first and second image data enables the detection of color changes of tissue such as the rectum “R” and colon “C” (see FIG. 3-5). These color changes indicate the presence and absence of blood filling the blood vessels within the tissue such as the rectum “R” and colon “C” (see FIG. 3-5) as the heart beats and, thus, can be evaluated in density and/or magnitude to determine a level of perfusion. While the pulsations and color changes are present, these the pulsations and color changes may be minute and, thus, difficult to detect; accordingly, in aspects as noted above, amplification may be utilized to facilitate detection of these pulsations and color changes. Alternatively or additionally, a machine learning algorithm 708 may be utilized to facilitate determination of a level of perfusion, as detailed below with reference to FIG. 7.

Continuing with reference to FIG. 6, an output indicating the level of perfusion determined at 630 is ultimately output at 640. The determined level of perfusion may be, for example, a categorical rating (for example: good, adequate, or poor), a relative metric (e.g., a percentage of detected perfusion compared to a baseline), or any other suitable indication of a level of perfusion. The output may include a visual, audible, and/or tactile output indicating the determined level of perfusion. The output may include an indicator that provides the determined level of perfusion itself, e.g., the categorical rating or relative metric, and/or that represents the determined level of perfusion, e.g., where the level, intensity, size, color, volume, pattern, etc. of the indicator indicates the determined level of perfusion. Alternatively or additionally, the output may only be provided, e.g., as a visual, audible, and/or tactile warning or alert indicator, if the level of perfusion is of a certain category (e.g., poor) or crosses a threshold (e.g., less than 50% of the baseline).

Referring to FIG. 7, in aspects, determining the level of perfusion (e.g., at 630 in method 600 of FIG. 6), is facilitated using a machine learning algorithm 708. More specifically, the image data 702 is provided as an input to the machine learning algorithm 708. The image data 702 may be the first and second image data and/or image data corresponding to the differences between the first and second image data (whether or not pre-processed, e.g., amplified). Additional data 706 may also be input to machine learning algorithm 708. The additional data 706 may include, for example: locations and/or types of identified tissue structures (e.g., rectum “R” and/or colon “C” (FIGS. 3-5)); locations and/or types of completed surgical tasks (e.g., an anastomosis “A” (FIGS. 3-5)); a type of surgical procedure (e.g., an LAR); status of the surgical procedure (e.g., pre-anastomosis or post-anastomosis); patient demographic information; patient health information (health conditions, blood pressure, heart rate, etc.); a catalogue of known tissue structures including expected perfusion, and/or blood vessel locations/densities thereof; and/or information pertaining to the instruments and/or surgical techniques utilized in the surgical procedure. Other suitable additional data 706 is also contemplated.

Based on the input data 702, 706, the machine learning algorithm 708 determines, as an output 710, a level of perfusion. The machine learning algorithm 708 may implement one or more of: supervised learning, semi-supervised learning, unsupervised learning, reinforcement learning, association rule learning, decision tree learning, anomaly detection, feature learning, computer vision, etc., and may be modeled as one or more of a neural network, Bayesian network, support vector machine, genetic algorithm, etc. The machine learning algorithm 708 may be trained based on empirical data and/or other suitable data and may be trained prior to deployment for use during a surgical procedure or may continue to learn based on usage data after deployment and use in a surgical procedure(s).

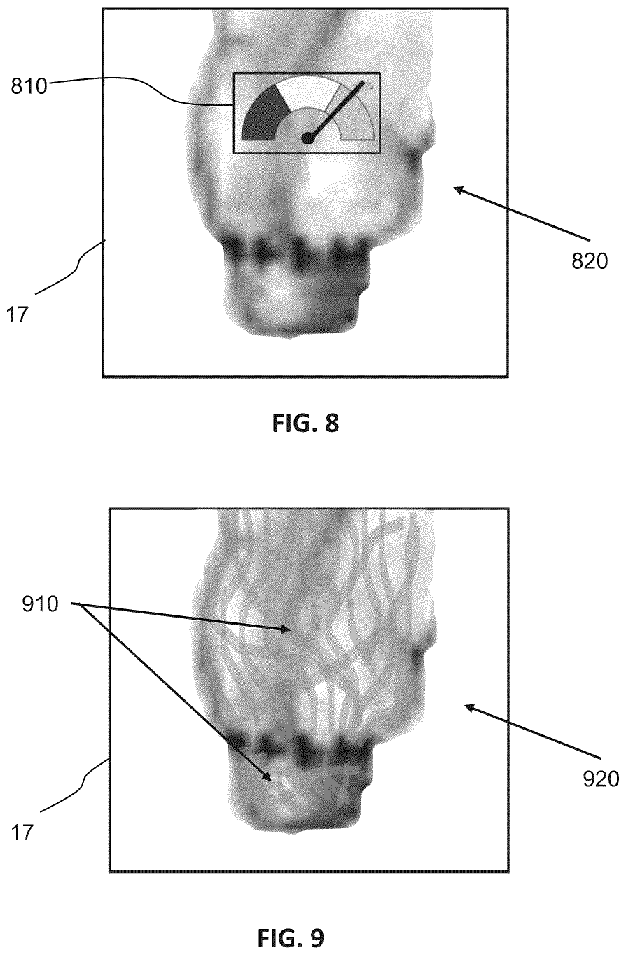

Referring to FIG. 8, as noted above, the determined level of perfusion may be a categorical rating (for example: good, adequate, or poor), a relative metric (e.g., a percentage of detected perfusion compared to a baseline), or any other suitable indication of the determined level of perfusion. The corresponding output based on the determined level of perfusion may be, for example, an indicator 810 in the form of a gauge overlaid or otherwise displayed on display 17, e.g., in connection with a video feed 820 of the surgical site. An overall output indicative of the determine level of perfusion may be provided; additionally or alternatively, different outputs may be provided for different tissue and/or portions of tissue (wherein the outputs are disposed on the corresponding tissues or tissue portions or are otherwise associated therewith). As an alternative to a gauge, indicator 810 may include one or more icons, symbols, text, combinations thereof, etc. indicating the determined level of perfusion. Indicator 810 may also include highlights (in color, shade, pattern, intensity, etc.) of tissue (and/or different tissues and/or portions of tissue) corresponding to the determined level of perfusion thereof overlaid on those tissues or portions thereof on video feed 820. As such, the level of perfusion of the tissues or portions can be determined, relative to the baseline and/or relative to one another.

Regardless of the particular configuration of indicator 810, method 600 (FIG. 6) may be repeated (repeatedly running machine learning algorithm 708 (FIG. 7), for example) such that the level of perfusion is repeatedly determined and indicator 810 is repeatedly updated. This may be done upon user-request, periodically, or continuously, e.g., in real-time.

Turning to FIG. 9, in aspects, in addition or as an alternative to outputting an indicator associated with a determined level of perfusion, perfusion information 910 may be displayed on display 17, e.g., in connection with a video feed 920 of the surgical site. Perfusion information 910 may include, for example, the ultraviolet-enhanced image data and/or data representing the amplified differences between the first and second image data. This perfusion information 910 may be overlaid on corresponding tissues or portions thereof on video feed 920. As such, the level of perfusion of the tissues or portions thereof can be more readily ascertained than from the video feed 920 alone.

It is understood that the various aspects disclosed herein may be combined in different combinations than the combinations specifically presented hereinabove and in the accompanying drawings. In addition, while certain aspects of the present disclosure are described as being performed by a single module or unit for purposes of clarity, it is understood that the techniques of this disclosure may be performed by a combination of units or modules associated with, for example, a surgical system.

Accordingly, although several aspects and features of of the disclosure are shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular aspects and features. Those skilled in the art will envision other modifications within the scope and spirit of the claims appended hereto.

What is claimed is:

1. A surgical system for detecting perfusion, comprising:
at least one surgical camera configured to obtain image data of tissue at a surgical site, the image data including first image data and second image data, the second image data temporally-spaced relative to the first image data; and
a computing device configured to receive the image data from the at least one surgical camera, the computing device including a non-transitory computer-readable storage medium storing instructions configured to cause the computing device to:
detect differences between the first and second image data;
determine a level of perfusion in the tissue based on the detected differences between the first and second image data; and
provide an output indicative of the determined level of perfusion in the tissue.

2. The surgical system according to claim 1, wherein the computing device is further caused to amplify the detected differences between the first and second image data and wherein the level of perfusion in the tissue is determined based on the amplified detected differences between the first and second image data.

3. The surgical system according to claim 1, wherein the at least one surgical camera includes first and second surgical cameras, and wherein the image data is stereographic image data from the first and second surgical cameras.

4. The surgical system according to claim 1, further comprising an ultraviolet light source configured to illuminate the tissue at the surgical site, wherein the image data includes ultraviolet-enhanced image data.

5. The surgical system according to claim 1, wherein the image data is video image data, infrared image data, thermal image data, or ultrasound image data.

6. The surgical system according to claim 1, wherein the level of perfusion is determined by a machine learning algorithm of the computing device.

7. The surgical system according to claim 6, wherein the machine learning algorithm is configured to receive the detected differences between the first and second image data and determine the level of perfusion based on the detected differences between the first and second image data.

8. The surgical system according to claim 6, wherein the machine learning algorithm is configured to receive the first and second image data, to detect the differences between the first and second image data, and to determine the level of perfusion based on the detected differences between the first and second image data.

9. The surgical system according to claim 1, wherein the output indicative of the determined level of perfusion in the tissue includes a visual indicator on a display configured to display a video feed of the surgical site.

10. The surgical system according to claim 1, wherein the output indicative of the determined level of perfusion in the tissue includes a visual overlay, on a display, over a video feed of the surgical site.

11. A method for detecting perfusion in surgery, comprising:
obtaining, from at least one surgical camera, first image data of tissue at a surgical site;
obtaining, from the at least one surgical camera, second image data of the tissue at the surgical site, the second image data temporally-spaced relative to the first image data;
detecting differences between the first and second image data;
determining a level of perfusion based on the detected differences between the first and second image data; and
providing an output indicative of the determined level of perfusion.

12. The method according to claim 11, further comprising amplifying the detected differences between the first and second image data before determining the level of perfusion in the tissue, and wherein the level of perfusion in the tissue is determined based on the amplified detected differences between the first and second image data.

13. The method according to claim 11, wherein obtaining each of the first and second image data includes obtaining, from first and second surgical cameras, the first image data as first stereographic image data and the second image data as second stereographic image data, respectively.

14. The method according to claim 11, further comprising illuminating the tissue at the surgical site with ultraviolet light, wherein the first image data is ultraviolet-enhanced image data, and wherein the second image data is ultraviolet-enhanced image data.

15. The method according to claim 11, wherein obtaining the first image data includes obtaining first video image data, first infrared image data, first thermal image data, or first ultrasound image data, and wherein obtaining the second image data includes obtaining second video image data, second infrared image data, second thermal image data, or second ultrasound image data.

16. The method according to claim 11, wherein determining the level of perfusion based on the detected differences between the first and second image data includes implementing a machine learning algorithm.

17. The method according to claim 16, wherein the machine learning algorithm is configured to receive the detected differences between the first and second image data and determine the level of perfusion based on the detected differences between the first and second image data.

18. The method according to claim 16, wherein the machine learning algorithm is configured to receive the first and second image data, to detect the differences between the first and second image data, and to determine the level of perfusion based on the detected differences between the first and second image data.

19. The method according to claim 11, wherein providing the output indicative of the determined level of perfusion in the tissue includes providing a visual indicator on a display configured to display a video feed of the surgical site.

20. The method according to claim 11, wherein providing the output indicative of the determined level of perfusion in the tissue includes providing a visual overlay, on a display, over a video feed of the surgical site.
